(19) United States
(12) Patent Application Publication
(10) Pub. No.: US 2009/0306397 A1
(43) Pub. Date: Dec. 10, 2009

(54) Method for the production of substituted azoles

(76) Inventors: Rainer Bruns, Leverkusen (DE); Hermann Uhr, Leverkusen (DE); Erasmus Vogl, Leverkusen (DE)

Correspondence Address: LANXESS CORPORATION, 111 RIDC PARK WEST DRIVE, PITTSBURGH, PA, 15275-1112, US

(21) Appl. No.: 11/921,252

(22) PCT Filed: May 27, 2006
(86) PCT No.: PCT/EP2006/005092

§ 371 Date: Jan. 30, 2009

(30) Foreign Application Priority Data
Jun. 7, 2005 (DE) 10 2005 025 992.8

(51) Int. Cl. C07D 257/04

(52) U.S. Cl. 548/252; 548/250

(57) ABSTRACT

The novel process for preparing substituted azoles allows compounds of the general formula (I) and/or their salts and/or their acid addition compounds

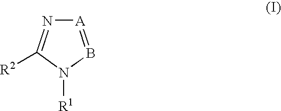

in which the substituents R¹ and R², A and B are as defined in the description to be prepared in good yield and in a simple, economically favourable manner.

The present invention relates to a novel process for preparing substituted azoles, in particular substituted 1H-tetrazoles and substituted 1H-triazoles.

Azoles, in particular the 5-substituted 1H-triazoles and -tetrazoles, are used inter alia as pharmaceutically active substances in medicine or are applied, for example, as biocides in crop protection and in the protection of industrial materials.

Starting materials for the synthesis of 5-substituted 1H-triazoles and -tetrazoles are usually the corresponding 5-H-substituted compounds. These are usually converted by lithiation at very low temperature and treatment with an electrophile into the corresponding 5-substituted derivatives. The following example serves to illustrate the closest prior art.

Thus, Yoshitaka Satoh and Nicholas Marcopulos describe a method [Tetrahedron Letters (1995), 36(11), 1759-62] for application of the lithiation of 1-benzyl- and 1-p-methoxybenzyltetrazoles at the 5-position. Reaction with n-butyllithium followed by treatment with electrophiles gave 5-functionalized 1-benzylic tetrazoles. However, this method has the disadvantages of an extremely low temperature (−98° C.) and the fact that n-BuLi is used.

In summary, it may be said that the lithiation, as closest prior art, is the method of choice for derivatizing the 5-position, for example with halogens, major disadvantages being the low temperature, the use of air-sensitive and expensive metallation reagents, such as n-BuLi, and in particular also the complete instability of the metallated intermediate even at temperatures above −78° C.

Accordingly, it was an object of the present invention to provide an improved process for preparing substituted azoles.

Surprisingly, we have now found a novel process for preparing substituted azoles which allows the lithiation described in the prior art and the practice of the reaction at low temperature to be avoided.

The present invention provides a process for preparing substituted azoles of the general formula (I) and/or their salts and/or their acid addition compounds

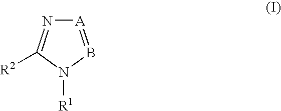

in which
A represents N, CH or CR³,
B represents N, CH or CR⁴,
with the proviso that at least one of the radicals A and B represents N,
R¹ represents hydrogen or represents in each case optionally substituted alkyl, alkenyl, alkynyl or phenyl,
R² represents F, Cl, Br, I, OH, SH, CN, SCN, or represents in each case optionally substituted alkyl, cycloalkyl, phenethyl, benzyl, acyl, thioacyl, hydroxymethylene or methylenethiol,
R³ represents in each case optionally substituted alkyl, alkenyl, alkynyl, phenyl or phenethyl, and
R⁴ represents in each case optionally substituted alkyl, alkenyl, alkynyl, phenyl or phenethyl,
   by reacting compounds of the formula (II) and/or their salts and/or their acid addition compounds

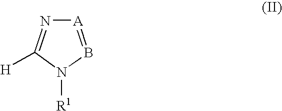

in which
A, B and R¹ are as defined for formula (I) above,
with at least one electrophile at a temperature between 0° C. and 100° C. in the presence of at least one base, if appropriate in the presence of a phase-transfer catalyst and if appropriate in the presence of a solvent or solvent mixture.

The process according to the invention preferably serves to prepare compounds of the general formula (I) in which
R¹ represents hydrogen or represents straight-chain or branched C₁-C₈-alkyl, C₂-C₈-alkenyl or C₂-C₈-alkynyl, each of which is optionally mono- or polysubstituted by identical or different substituents from the group consisting of halogen; nitro; cyano; hydroxyl; C₁-C₆-alkoxy which is optionally mono- to nonasubstituted by identical or different halogen substituents; C₁-C₆-alkylthio which is optionally mono- to nonasubstituted by identical or different halogen substituents; amino; monoalkylamino having straight-chain or branched C₁-C₆-alkyl radicals; dialkylamino having identical or different straight-chain or branched C₁-C₆-alkyl radicals; phenyl which is optionally mono- or polysubstituted by identical or different substituents from the group consisting of halogen, nitro, cyano, alkyl, haloalkyl, alkoxy, haloalkoxy, alkylthio, haloalkylthio, acyl, acyloxy, (alkoxy)carbonyl, carboxyl, amino, monoalkylamino or dialkylamino,
   or
R¹ represents phenyl which is optionally mono- or polysubstituted by identical or different substituents from the group consisting of halogen, nitro, cyano, hydroxyl, alkyl, haloalkyl, alkoxy, haloalkoxy, alkylthio, haloalkylthio, acyl, acyloxy, alkoxycarbonyl, carboxyl, amino, monoalkylamino, dialkylamino,
R² represents F, Cl, Br, I, OH, SH, CN, SCN, straight-chain or branched C₁-C₈-alkyl or C₁-C₁₀-cycloalkyl,
   or
   represents phenethyl or benzyl, each of which is optionally mono- or polysubstituted by identical or different substituents from the group consisting of halogen; nitro; cyano; hydroxyl; C₁-C₆-alkoxy which is optionally mono- to nonasubstituted by identical or different halogen substituents; C₁-C₆-alkylthio which is optionally mono- to nonasubstituted by identical or different halogen substituents; amino; monoalkylamino having straight-chain or branched C₁-C₆-alkyl radicals; dialkylamino having identical or different straight-chain or branched C₁-C₆-alkyl radicals; phenyl which is optionally mono- or polysubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino or dialkylamino,
   or
   represents acyl or thioacyl, each of which is optionally substituted by hydroxyl; thiohydroxyl; straight-chain or branched C₁-C₈-alkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino, dialkylamino; C₁-C₁₀-cycloalkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino, dialkylamino; phenyl which is optionally mono- or polysubstituted by identical or different substituents from the group consisting of halogen, nitro, cyano, alkyl, haloalkyl, alkoxy, haloalkoxy, alkylthio, haloalkylthio, acyl, acyloxy, (alkoxy)carbonyl, carboxyl, amino, monoalkylamino or dialkylamino,
   or
   represents hydroxymethylene or methylenethiol, each of which is optionally substituted by straight-chain or branched C₁-C₈-alkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, (alkoxy)carbonyl, carboxyl, amino, monoalkylamino, dialkylamino; C₁-C₁₀-cycloalkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino, dialkylamino; phenyl which is optionally mono- or polysubstituted by identical or different substituents from the group consisting of halogen, nitro, cyano, alkyl, haloalkyl, alkoxy, haloalkoxy, alkylthio, haloalkylthio, acyl, acyloxy, (alkoxy)carbonyl, carboxyl, amino, monoalkylamino or dialkylamino,
A represents N, CH or CR³,
   where
R³ represents straight-chain or branched C₁-C₈-alkyl, C₂-C₈-alkenyl or C₂-C₈-alkynyl, each of which is optionally mono- or polysubstituted by identical or different substituents from the group consisting of halogen, nitro, cyano, hydroxyl, alkylthio, alkoxy, amino, and
B represents N, CH or CR⁴,
   where
R⁴ represents straight-chain or branched C₁-C₈-alkyl, C₂-C₈-alkenyl or C₂-C₈-alkynyl, each of which is optionally mono- or polysubstituted by identical or different substituents from the group consisting of halogen, nitro, cyano, hydroxyl, alkylthio, alkoxy, amino,
   with the proviso that at least one of the radicals A and B represents N.

Particularly preferably, the process according to the invention serves to prepare compounds of the general formula (I) in which
R¹ represents hydrogen or represents straight-chain or branched C₁-C₈-alkyl, C₂-C₆-alkenyl or C₂-C₆-alkynyl, each of which is optionally mono- to tetrasubstituted by identical or different substituents from the group consisting of fluorine; chlorine; bromine; nitro; cyano; hydroxyl; C₁-C₄-alkoxy which is optionally mono- to pentasubstituted by identical or different substituents from the group consisting of fluorine, chlorine and bromine; C₁-C₄-alkylthio which is optionally mono- to pentasubstituted by identical or different substituents from the group consisting of fluorine, chlorine or bromine; amino; monoalkylamino having straight-chain or branched C₁-C₄-alkyl radicals; dialkylamino having identical or different straight-chain or branched C₁-C₄-alkyl radicals; phenyl which is optionally mono- to tetrasubstituted by identical or different substituents from the group consisting of fluorine, chlorine, bromine, nitro, cyano, hydroxyl, C₁-C₄-alkyl, C₁-C₄-haloalkyl which is mono- to pentasubstituted by identical or different substituents from the group consisting of fluorine, chlorine and bromine, C₁-C₄-alkoxy, C₁-C₄-haloalkoxy which is mono- to pentasubstituted by identical or different substituents from the group consisting of fluorine, chlorine and bromine, C₁-C₄-alkylthio, C₁-C₄-haloalkylthio which is mono- to pentasubstituted by identical or different substituents from the group consisting of fluorine, chlorine and bromine, C₁-C₆-acyl, C₁-C₆-acyloxy, C₁-C₆-alkoxycarbonyl, carboxyl, amino, monoalkylamino having straight-chain or branched C₁-C₄-alkyl radicals, or dialkylamino having identical or different straight-chain or branched C₁-C₄-alkyl radicals,
   or
R¹ represents phenyl which is optionally mono- to tetrasubstituted by identical or different substituents from the group consisting of fluorine; chlorine; bromine; nitro; cyano; hydroxyl; C₁-C₄-alkyl; C₁-C₄-haloalkyl which is mono- to pentasubstituted by identical or different substituents from the group consisting of fluorine, chlorine and bromine; C₁-C₄-alkoxy; C₁-C₄-haloalkoxy which is mono- to pentasubstituted by identical or different substituents from the group consisting of fluorine, chlorine and bromine; C₁-C₄-alkylthio; C₁-C₄-haloalkylthio which is mono- to pentasubstituted by identical or different substituents from the group consisting of fluorine, chlorine and bromine; C₁-C₄-acyl; C₁-C₄-acyloxy; C₁-C₄-alkoxycarbonyl; carboxyl; amino; monoalkylamino having straight-chain or branched C₁-C₄-alkyl radicals, or dialkylamino having identical or different straight-chain or branched C₁-C₄-alkyl radicals,
R² represents F, Cl, Br, I, OH, SH, CN, SCN, straight-chain or branched C₁-C₁₀-alkyl or C₁-C₁₀-cycloalkyl,
   or
   represents benzyl which is optionally mono- or polysubstituted by identical or different substituents from the group consisting of halogen, nitro, cyano, hydroxyl, alkoxy, amino, monoalkylamino and dialkylamino,
   or
   represents acyl or thioacyl, each of which is optionally substituted by hydroxyl; thiohydroxyl; straight-chain or branched C₁-C₈-alkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino and dialkylamino; C₁-C₁₀-cycloalkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino and dialkylamino; or phenyl which is optionally mono- or polysubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino and dialkylamino,
   or
   represents hydroxymethylene or methylenethiol, each of which is optionally substituted by straight-chain or branched C₁-C₈-alkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino and dialkylamino; C₁-C₁₀-cycloalkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino and dialkylamino; or phenyl which is optionally mono- or polysubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino and dialkylamino,
A represents N, CH or CR³,
   where
R³ represents straight-chain or branched C₁-C₈-alkyl, C₂-C₈-alkenyl or C₂-C₈-alkynyl, each of which is optionally mono- or polysubstituted by identical or different substituents from the group consisting of halogen, nitro, cyano, hydroxyl, alkylthio, alkoxy and amino,
   and
B represents N, CH or CR⁴,
   where
R⁴ represents straight-chain or branched C₁-C₈-alkyl, C₂-C₈-alkenyl or C₂-C₈-alkynyl, each of which is optionally mono- or polysubstituted by identical or different substituents from the group consisting of halogen, nitro, cyano, hydroxyl, alkylthio, alkoxy and amino,
   with the proviso that at least one of the radicals A and B represents N.

Very particularly preferably, the process according to the invention serves to prepare compounds of the formula (I) in which
R¹ represents hydrogen, methyl, ethyl, n-propyl, isopropyl, n-butyl, isobutyl, sec-butyl, tert-butyl, n-pentyl, n-hexyl, n-heptyl, n-octyl, allyl, vinyl, propargyl, where the alkyl radicals mentioned are in each case optionally mono- to tetrasubstituted by identical or different substituents from the group consisting of fluorine, chlorine, bromine, nitro, cyano, hydroxy, methoxy, ethoxy, n-propoxy, isopropoxy, n-butoxy, isobutoxy, sec-butoxy, tert-butoxy, trifluoromethoxy, methylthio, ethylthio, n-propylthio, isopropylthio, trifluoromethylthio, amino, methylamino, ethylamino, n-propylamino, isopropylamino, dimethylamino, diethylamino, methylethylamino, di-n-propylamino, diisopropylamino, or by phenyl which is optionally mono- to trisubstituted by fluorine, chlorine, bromine, nitro, cyano, hydroxyl, methyl, ethyl, n-propyl, isopropyl, n-butyl, isobutyl, s-butyl, tert-butyl, trifluoromethyl, methoxy, ethoxy, n-propoxy, isopropoxy, n-butoxy, isobutoxy, sec-butoxy, tert-butoxy, trifluoromethoxy, methylthio, ethylthio, n-propylthio, isopropylthio, trifluoromethylthio, formyl, acetyl, acetyloxy, methoxycarbonyl, ethoxycarbonyl, carboxyl, amino, methylamino, ethylamino, n-propylamino, isopropylamino, dimethylamino, diethylamino, methylethylamino, di-n-propylamino or diisopropylamino,
   or
R¹ represents phenyl which is optionally mono- to trisubstituted by fluorine, chlorine, bromine, nitro, cyano, hydroxyl, methyl, ethyl, n-propyl, isopropyl, n-butyl, isobutyl, s-butyl, tert-butyl, trifluoromethyl, methoxy, ethoxy, n-propoxy, isopropoxy, n-butoxy, isobutoxy, sec-butoxy, tert-butoxy, trifluoromethoxy, methylthio, ethylthio, n-propylthio, isopropylthio, trifluoromethylthio, formyl, acetyl, acetyloxy, methoxycarbonyl, ethoxycarbonyl, carboxyl, amino, methylamino, ethylamino, n-propylamino, isopropylamino, dimethylamino, diethylamino, methylethylamino, di-n-propylamino, diisopropylamino,
R² represents F, Cl, Br, I, OH, SH, CN, SCN,
   or
   represents benzyl, hydroxymethylene or methylenethiol, each of which is optionally substituted by straight-chain or branched C₁-C₁₀-alkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino, dialkylamino; C₁-C₁₀-cycloalkyl which is optionally mono- to nonasubstituted by identical or different substituents from the group consisting of halogen, nitro, cyano, alkyl, haloalkyl, alkoxy, haloalkoxy, alkylthio, haloalkylthio, acyl, acyloxy, alkoxycarbonyl, carboxyl, amino, monoalkylamino, dialkylamino; or phenyl which is optionally mono- or polysubstituted by identical or different substituents from the group consisting of halogen, nitro, cyano, alkyl, haloalkyl, alkoxy, haloalkoxy, alkylthio, haloalkylthio, acyl, acyloxy, (alkoxy)carbonyl, carboxyl, amino, monoalkylamino and dialkylamino,
A represents N or CH,
   and
B represents N or CH,
   with the proviso that at least one of the radicals A and B represents N.

The process according to the invention also serves to prepare salts and/or acid addition compounds of compounds of the formula (I), such as, for example, their hydrohalides, hydrophosphonates or hydrosulphates, it being possible, for example, to employ the corresponding salts and/or acid addition compounds of the formula (II).

For carrying out the process according to the invention, compounds of the formula (II) are used. With respect to their general, preferred, particularly preferred and very particularly preferred meaning, the substituents R1, A and B in formula (II) correspond to the corresponding meanings, listed above, of the substituents R1, A and B in formula (I).

Suitable electrophiles for carrying out the process according to the invention are, for example, halogens, such as fluorine, chlorine, bromine, iodine, aldehydes, such as, for example, benzaldehyde, cyclohexanecarbaldehyde, nitrites, such as cyclohexanecarbonitrile, or amides, such as, for example, Weinreb amide. Preferred for use as electrophiles are chlorine, bromine, iodine and aldehydes, or mixtures thereof. The electrophiles are generally employed in amounts of from 0.5 to 15 equivalents, based on the azole (II). Preference is given to using 1 to 5 equivalents and in particular 1.1 to 3 equivalents of electrophile, based on the azole.

The process according to the invention is generally carried out at temperatures between 0° C. and 100° C., preferably between 15° C. and 80° C. and particularly preferably between 20° C. and 50° C.

Suitable solvents are all customary organic solvents which are not affected or cannot be decomposed by the strong basic environment, such as, for example, petroleum ether, n-octane, n-pentane, n-hexane, cyclohexane, toluene, benzene, THF, diethyl ether, methyl t-butyl ether, diglyme, methanol, ethanol, isopropanol, n-butanol, CH₂Cl₂, CHCl₃. It is also possible to use mixtures of two or more solvents. Depending on the solvent, monophasic or biphasic systems are obtained. In certain cases, it may also be possible to carry out the reaction completely without added organic solvent, the organic phase being formed by the substrate. Preference is given to using toluene, n-hexane, cyclohexane, diethyl ether, methyl t-butyl ether or THF.

Suitable bases for carrying out the process according to the invention are, for example, alkali metal hydroxides, phosphates, alkoxides and carbonates, and also mixtures thereof. Particularly suitable are, from the group of the alkali metal hydroxides, NaOH and KOH and, from the group of the carbonates, Cs₂CO₃, CaCO₃, MgCO₃. Very particular preference is given to using aqueous solutions of NaOH and/or KOH, preferably a 20% strength to 60% strength aqueous NaOH solution, particularly preferably an aqueous 30% strength to 55% strength NaOH solution. The base is employed in excess, based on the substrate, preferably 1 to 100 equivalents, particularly preferably 10 to 60 equivalents, per equivalent of substrate.

The process according to the invention can be carried out in the presence of a phase-transfer catalyst or other surface-active additives. Examples of suitable phase-transfer catalysts are 15-crown-5,18-crown-6, tetrabutylammonium hydrogensulphate, tetrabutylammonium bromide, tetrabutylammonium chloride, tetraoctylammonium bromide, tetraoctylammonium chloride, methyltridecylammonium chloride, methyltrioctylammonium chloride (Aliquat 336) and methyltributylammonium chloride. Preference is given to methyltrioctylammonium chloride (Aliquat 336) and methyltributylammonium chloride or mixtures thereof.

Examples of surface-active additives are molecular sieves, silica gel or alumina powders.

The phase-transfer catalyst can be employed in an amount of from 0.01 to 5 mol %, preferably from 0.3 to 3 mol %, based on the substrate.

The reaction can be carried out at various stirrer speeds which ensure that the reactants are mixed well. A stirrer speed of more than 10 000 rotations per minute, as is obtained, for example, when an Ultra-Turrax is used, may be advantageous here, but is not in all cases necessary.

Advantageously, the reaction can also be carried out in an ultrasonic bath.

The solution or suspension of the starting material is advantageously stirred with the basic solution for some time, the electrophile (if appropriate dissolved in a suitable solvent) is then metered in at a suitable rate and the mixture is stirred for some additional time. The optimum conditions depend on the substrate and its reactivity and solubility and have to be determined for each case; however, they are usually in the range of a few minutes or hours.

Compared to the processes hitherto employed, the procedure according to the invention has a number of advantages. It can be carried out in very cheap solvents. Depending on the substrate, the reaction does not require cooling or heating. Room temperature may be the most favourable temperature. The reaction proceeds quickly. The bases used are very inexpensive and readily available. The reagents used, such as, for example, bromine or iodine, are readily available. Control and optimization of the reaction is possible by a suitable dosage of the reactants and the choice of solvents. The reaction is readily transferable to a larger scale. In some examples, the product is formed in high yield and purity and does not have to be purified any further.

The following examples according to the invention may serve for illustration:

### EXAMPLES

### 1. Synthesis of 1-benzyl-5-iodo-1H-tetrazole

0.50 g of 1-benzyl-1H-tetrazole is suspended in 10 ml of hexane, 10 ml of 50% strength aqueous sodium hydroxide solution are added and the mixture is stirred thoroughly. Over a period of 15 minutes, 1.18 g of iodine, dissolved in 10 ml of tetrahydrofuran, are added dropwise. After the reaction has ended, the mixture is transferred into a separating funnel, the aqueous phase is extracted with ethyl acetate and the combined organic phases are washed with water and brine and dried with sodium sulphate. Removal of the solvent gives beige crystals which are purified on a silica gel column (mobile phase: ethyl acetate/n-hexane 35/65), affording 0.60 g of pure product (melting point 122° C., yield 67%).

### 2. Synthesis of 1-benzyl-5-iodo-1H-tetrazole

10 g of 1-benzyl-1H-tetrazole are dissolved in 200 ml of THF, 200 ml of 50% strength aqueous sodium hydroxide solution are added and the mixture is stirred thoroughly. Over a period of 15 minutes, 23 g of iodine, dissolved in tetrahydrofuran, are added dropwise. After reaction has ended, the mixture is transferred into a separating funnel, the aqueous phase is extracted with ethyl acetate and the combined organic phases are washed with water and brine and dried with sodium sulphate. Removal of the solvent gives beige crystals, 16.2 g of pure product (melting point 122° C., yield 91%).

### 3. Synthesis of 1-benzyl-5-bromo-1H-tetrazole

1.00 g of 1-benzyl-1H-tetrazole is dissolved in 10 ml of toluene, 10 ml of 50% strength aqueous sodium hydroxide solution are added and the mixture is stirred thoroughly. Over a period of 15 minutes, 2.37 g of bromine, dissolved in 10 ml of toluene, are added dropwise. After the reaction has ended, the mixture is transferred into a separating funnel, the aqueous phase is extracted with ethyl acetate and the combined organic phases are washed with water and brine and dried with sodium sulphate. Removal of the solvent and purification on a silica gel column gives 1.09 g of pure product (melting point 54° C., yield 73%).

### 4. Synthesis of 1-n-octyl-5-iodo-1H-tetrazole

0.50 g of n-octyl-1H-tetrazole is dissolved in 10 ml of THF, 10 ml of 50% strength aqueous sodium hydroxide solution are added and the mixture is stirred thoroughly. Over a period of 15 minutes, 1.04 g of iodine, dissolved in 10 ml of tetrahydrofuran, are added dropwise, and the mixture is stirred for another hour. After the reaction has ended, the mixture is transferred into a separating funnel, the aqueous phase is extracted with ethyl acetate and the combined organic phases are washed with water and brine and dried with sodium sulphate. Purification gives 0.19 g of pure product which crystallizes on standing (melting point 40° C., yield 22%).

### 5. Synthesis von 1-n-octyl-5-iodo-1H-tetrazole

0.50 g of n-octyl-1H-tetrazole is dissolved in 10 ml of toluene, 10 ml of 50% strength aqueous sodium hydroxide solution is added and the mixture is stirred thoroughly. Over a period of 15 minutes, 1.04 g of iodine, suspended in 10 ml of toluene, are added dropwise, and the mixture is stirred for another hour. After the reaction has ended, the mixture is transferred into a separating funnel, the aqueous phase is extracted with ethyl acetate and the combined organic phases are washed with water and brine and dried with sodium sulphate. Purification gives 0.17 g of pure product which crystallizes on standing (melting point 40° C., yield 20%).

### 6. Synthesis of 1-n-octyl-5-iodo-1H-tetrazole

0.50 g of n-octyl-1H-tetrazole is dissolved in 10 ml of toluene, 10 ml of 50% strength aqueous sodium hydroxide solution and 0.02 ml Aliquat are added and the mixture is stirred thoroughly. Over a period of 15 minutes, 1.04 g of iodine, suspended in 10 ml of toluene, are added dropwise, and the mixture is stirred for another hour. After the reaction has ended, the mixture is transferred into a separating funnel, the aqueous phase is extracted with ethyl acetate and the combined organic phases are washed with water and brine and dried with sodium sulphate. Purification gives 0.30 g of pure product which crystallizes on standing (melting point 40° C., yield 35%).

### 7. Synthesis of (1-benzyl-1H-tetrazol-5-yl)(phenyl)methanol

1.00 g of 1-benzyl-1H-tetrazole is dissolved in 10 ml of THF, 10 ml of 50% strength aqueous sodium hydroxide solution is added and the mixture is stirred thoroughly for 1 hour. Over a period of 15 minutes, 0.944 g of benzaldehyde, dissolved in 10 ml of THF, is added dropwise. After the reaction has ended, the mixture is transferred into a separating funnel, the aqueous phase is extracted with ethyl acetate and the combined organic phases are washed with water and brine and dried with sodium sulphate. Removal of the solvent and purification on a silica gel column gives 0.60 g of pure product (melting point 81° C., yield 36%).

### 8. Synthesis of (1-benzyl-1H-tetrazol-5-yl)(cyclohexyl)methanol

5.6 g of 1-benzyl-1H-tetrazole are dissolved in 10 ml of THF, 10 ml of 50% strength aqueous sodium hydroxide solution are added and the mixture is stirred thoroughly for 1 hour. Over a period of 15 minutes, 5.6 g of cyclohexanealdehyde, dissolved in 10 ml of THF, are added dropwise. After 20 more minutes of stirring, once the reaction has ended, the mixture is transferred into a separating funnel, the aqueous phase is extracted with ethyl acetate and the combined organic phases are washed with water and brine and dried with sodium sulphate. Removal of the solvent and purification on a silica gel column gives 3.0 g of a colourless viscous oil (R_{f} 1.5056, yield 32%).

1. A process for preparing at least one substituted azol of the general formula (I) and/or salt thereof and/or an acid addition compound thereof 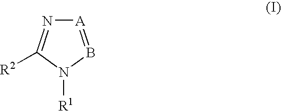
in which
A represents N, CH or CR³,
B represents N, CH or CR⁴,
with the proviso that at least one of the radicals A or B represents N,
R¹ represents hydrogen or represents in each case optionally substituted alkyl, alkenyl, alkynyl or phenyl,
R² represents F, Cl, Br, I, OH, SH, CN, SCN, or represents in each case optionally substituted alkyl, cycloalkyl, phenethyl, benzyl, acyl, thioacyl, hydroxymethylene or methylenethiol,
R³ represents in each case optionally substituted alkyl, alkenyl, alkynyl, phenyl or phenethyl, and
R⁴ represents in each case optionally substituted alkyl, alkenyl, alkynyl, phenyl or phenethyl,
comprising reacting a compound of the formula (II) and/or a salt and/or an acid addition compound thereof 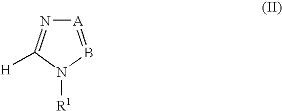
where A, B and R¹ are as defined for formula (I) above,
with at least one electrophile at a temperature between 0° C. and 100° C. in the presence of at least one base.

2. The process according to claim 1, wherein the electrophile is a compound selected from the group consisting of halogens, aldehydes, nitriles, amides, and mixtures thereof.

3. The process according to claim 1, wherein the electrophile is employed in an amount of from 0.5 to 15 equivalents, based on the amount of the compound of the formula (II).

4. The process according to claim 1, wherein the base is an alkali metal hydroxide, a phosphate, an alkoxide, a carbonate or a mixture thereof and is employed in an amount of from 1 to 100 equivalents based on the amount of the compound of the formula (II).

5. The process according to claim 8, wherein the phase-transfer catalyst is a compound selected from the group consisting of 15-crown-5, 18-crown-6, tetrabutylammonium hydrogensulphate, tetrabutylammonium bromide, tetrabutylammonium chloride, tetraoctylammonium bromide, tetraocylammonium chloride, methyltridecylammonium chloride, methyltrioctylammonium chloride (Aliquat 336), methyltributylammonium chloride, and a mixture thereof.

6. The process according to claim 8, wherein the phase-transfer catalyst is present in an amount of from 0.01 to 5 mol %, based on the amount of the compound of the formula (II).

7. The process according to claim 11, wherein the solvent or the solvent mixture present is toluene, n-hexane, cyclohexane, diethyl ether, methyl t-butyl ether, THF or a mixture thereof.

8. The process according to claim 1, wherein said reacting is performed in the presence of a phase-transfer catalyst.

9. The process according to claim 1, wherein said reacting is performed in the presence of a surface-active additive.

10. The process according to claim 9, wherein said surface-active additive is a molecular sieve, silica gel or an alumina powder.

11. The process according to claim 1, wherein said reacting is performed in the presence of a solvent or a solvent mixture.
